(19) United States
(12) Patent Application Publication
(10) Pub. No.: US 2003/0135141 A1
(43) Pub. Date: Jul. 17, 2003

(54) Orthesis for treatment of injuries to the acromioclavicula shoulder joint

(76) Inventor: Gunther Berhorst, Wehretal (DE)

Correspondence Address: COLLARD & ROE, P.C., 1077 Northern Boulevard, Roslyn, NY, 11576-1696, US

(21) Appl. No.: 10/317,028

(22) Filed: Dec. 11, 2002

(30) Foreign Application Priority Data
Dec. 11, 2001 (DE) 201 20 016.3

(51) Int. Cl. A61F 5/00

(52) U.S. Cl. 602/4; 602/20

(57) ABSTRACT

An orthesis for the treatment of injuries to the acromioclavicula joint, especially for the treatment of Tossy I/II/III injuries, has three straps positioned on a shoulder pad. The first strap and the second strap are secured to a forearm holder to be positioned in front of the elbow joint. The first strap can be placed in front of the shoulder and the second strap can be placed behind the shoulder. The third strap is designed to run behind the back to the opposite shoulder and has a loop to be fixed there. Pressure can thus be exerted on the clavicle and at the same time the shoulder can be pulled upwards.

### BACKGROUND

The invention relates to an orthesis for treating injuries to the acromioclavicula joint (A-C joint) with a shoulder pad and straps connected to the shoulder pad.

An apparatus is disclosed in German utility model G 92 15 341.0 wherein a shoulder shell is provided to stabilize one shoulder of a patient and is held there with straps. German utility model DE 201 14 446 U1 discloses a shoulder-joint function bandage which is especially designed for the treatment of hemiparesis patients.

Apparatuses for post-operative application for the restoration of the upper arm and the shoulder are shown in DE 41 34 969 A1 and German utility model DE 90 10 801.9.

The laceration of and injury to the ligament structures of the acromioclavicula shoulder joint (A-C joint) are frequent clinical occurrences in surgery and orthopaedics. Treatment involves immobilizing the joint with bandages. Bandages of various designs are already known, most involve considerable movement restriction of the injured shoulder and frequently the entire arm.

Laceration of the ligament structures in the A-C joint involves raising of the clavicle and the collar bone, the so-called piano-key phenomenon. The applied bandage should allow the structures to join together again and heal together. Therefore pressure is applied to the clavicle in order to press it in the direction of the acromium or shoulder. However, this is frequently unsuccessful. After weeks of bandage treatment the patient must, under certain circumstances, undergo an operation to regain the function of the shoulder joint. The healing of the injury is thereby be delayed.

The object of the invention is to provide an orthesis which achieves sufficient stability of the injured shoulder and fixes the joint securely while maintaining a high freedom of movement for the patients.

### SUMMARY

In the present invention, it is important that at least three straps are placed on or connected to the shoulder pad. The first strap and the second strap are secured to a forearm holder positioned just below the elbow joint. The first strap can be placed in front of the shoulder and the second strap can be placed behind the shoulder. A third strap is constructed and/or is provided to run behind the back of the patient to the opposite shoulder and has a loop to fix it there.

Tossy I/II/III injuries in particular can be treated with this orthesis. However, it is also suitable for Rockwood type III to type VI injuries as well as lateral collar-bone or clavicula fractures. In this use pressure is applied downwards onto the clavicle while pulling the shoulder upwards at the same time. This joint comes into its normal position and the patient retains his natural freedom of movement to a large extent. This orthesis allows natural movements without endangering the aim of the therapy. The patient achieves a positive attitude regarding the injury positively influencing the healing process.

The shoulder pad has an anti-slip support, preferably silicone, on its side facing the user or the skin of the user. This prevents slippage of the shoulder pad and improves comfort. The shoulder pad is made of a textile material. In order to provide sufficient stability, the textile material is preferably impregnated with a polyurethane or can contain other suitable reinforcements, such as aluminium. The construction of the shoulder pad using plastic is also advantageous.

The straps are detachably connected to the shoulder pad. In the area of the shoulder pad, rings or eyes are provided through which the straps are guided and placed adjustably in length, fixable with a velcro closure. These rings may have comparatively short attachment straps.

The straps are secured at three clamping points separated from one another. The three clamping or holding points are arranged on the shoulder pad so that the straps are arranged at an angle of 120° with respect to one another. The straps are more favorably made of textile material or a hygienically safe plastic material.

The forearm holder is preferably constructed as a clasp completely encircling the forearm or as a forearm cuff. This has a double velcro closure arranged at each end, forming a ring encircling the forearm. Fundamentally the forearm holder could also be constructed as an upwardly open shell. The forearm holder and the shoulder pad are preferably made of the same textile material. In a preferred embodiment of the invention the orthesis is shaped so that the first strap and the second strap run in front of and behind the shoulder, starting from the shoulder pad, and crossing under the upper arm and being secured at the forearm holder. This creates increased stability of the shoulder pad. Preferably the first strap and the second strap are secured to the shoulder pad and to the forearm holder so that they cross below the upper arm. An anti-slip feature may be provided on the inside of the forearm holder. This is formed by a silicone insert, preferably with burls or another rough structure.

The loop provided at the end of the third strap can be opened with a velcro closure. In another embodiment the loop is closed and the strap arranged thereon is comparatively long so that the loop can be pulled simply over the arm. The length of the strap is variably adjustable by means of a guide at the shoulder pad and is secured by a velcro connection.

The orthesis has a first holding strap which secured to the wrist and dimensioned so that it is guided around the back in the elongation of the forearm bent in front of the body and is fixable on the upper arm of the injured shoulder. This can be accomplished either with a loop or with a velcro closure. By this means, the forearm is held in position and the wearing comfort is increased. In a further embodiment of the invention the orthesis is assigned a second holding strap secured to the wrist and designed and dimensioned so that it is guided upwards starting from the forearm bent in front of the body and secured on the third strap in the area of the shoulder opposite the injury. This holding strap is also used to fix the forearm in front of the body and increases the wearing comfort of the orthesis. The two holding straps described can be used singly but preferably combined. One of the holding straps preferably has a wrist holder in the form of a wrist cuff and the other holding strap is fixable on this wrist holder. The first holding strap preferably has this wrist holder and a flap through which the second holding strap can pass.

### BRIEF DESCRIPTION OF THE DRAWINGS

Other objects and features of the present invention will become apparent from the following detailed description considered in connection with the accompanying drawings which disclose at least one embodiment of the present invention. It should be understood, however, that the drawings are designed for the purpose of illustration only and not as a definition of the limits of the invention.

In the drawings, wherein similar reference characters denote similar elements throughout the several views:

FIG. 1 is a schematic front view of a preferred embodiment of the orthesis according to the invention on a patient;

FIG. 2 is an overall schematic view of a preferred embodiment of the orthesis according to the invention;

FIG. 3 is a schematic view of the first holding strap assigned to the orthesis according to the invention;

FIG. 4 is a schematic view of the second holding strap assigned to the orthesis according to the invention;

FIG. 5 is a schematic front view of an embodiment of the orthesis according to the invention on a patient including the holding straps;

FIG. 6 is a rear view as in FIG. 5; and

FIG. 7 is an enlarged view of the shoulder region with slightly out-stretched arm.

### DETAILED DESCRIPTION

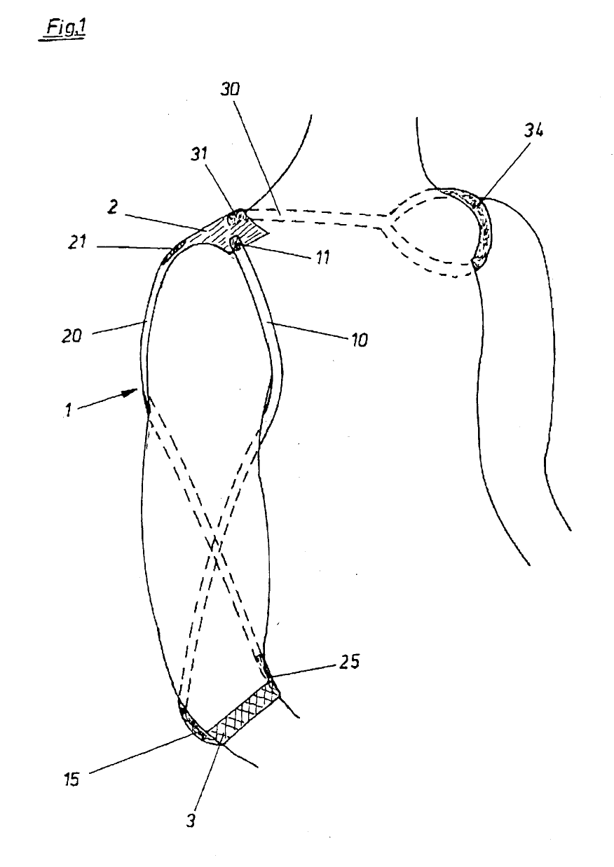

FIG. 1 shows an orthesis 1 according to the invention for the treatment of injuries to the A-C shoulder joint, especially Tossy I to III injuries. The orthesis has a shoulder pad 2, a forearm holder 3 and three straps 10, 20 and 30. Shoulder pad 2, made of one piece, is adapted to the anatomical conditions of the shoulder and has a skin-friendly silicone pad on its underside facing the skin. This guarantees an absolutely slip-proof fit during a lateral abduction of the arm up to approximately 85°, and further with a design having a back strap.

On the upper side of shoulder pad 2 are three holding points 11, 21, 31. Two holding points 11, 21 each receive straps 10, 20 which pass in front of and behind the shoulder in the direction of the elbow, crossing on the underside of the forearm. They are fixed on clamping points 15 and 25 to forearm holder 3, located on the forearm and holding on to the bony structures present there, and encircling the arm in a clasp fashion. Third holding point 31 on shoulder pad 2 serves to secure strap 30 which runs transversely over the back to the opposite shoulder and emerges there in a softly padded loop 34. Strap 30 is also designed as a velcro strip.

Shoulder pad 2 as well as forearm holder 3 can be made of plastic and textile material. When made of textile material, it is recommended that reinforcing elements be inserted, such as, shaped parts made of easily formable materials, such as aluminium. First strap 10 and second strap 20 preferably have a width of approximately 30 to 40 mm and are made of so-called luggage strap or similar material which best adapts to the anatomical shapes and conditions on account of its flexible quality. Third strap 30 is made of a softly padded material. Straps 10, 20, 30 and the loop are shown dashed where they run behind the arm or the back and are not visible when viewed in the perspective shown.

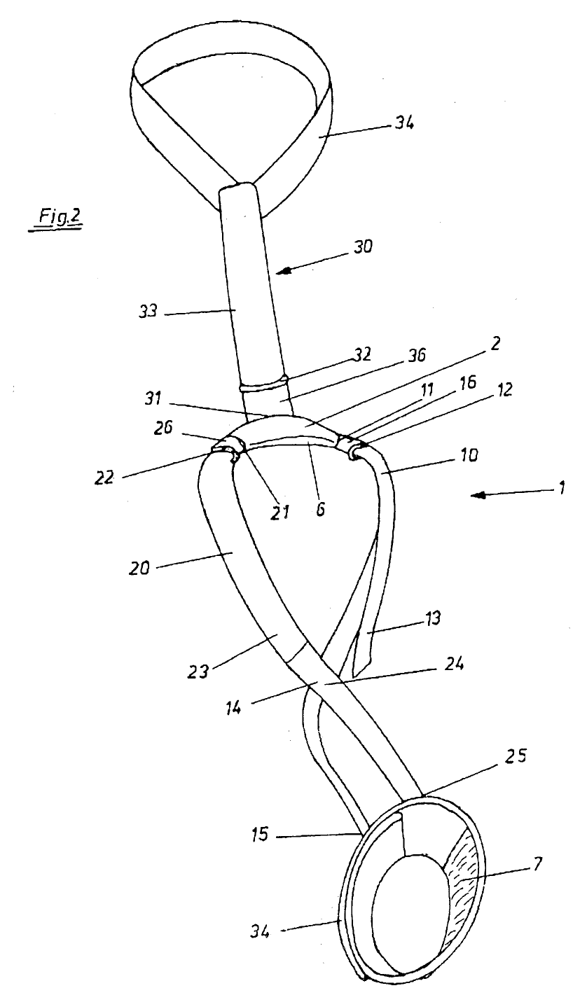

FIG. 2 shows orthesis 1 according to the invention with shoulder pad 2, straps 10, 20 and 30 and forearm holder 3. Shoulder pad 2 has a substantially oval basic shape. However, on the side facing the shoulder there is a circular segment or sickle-shaped recess 6. First strap 10 and second strap 20 are placed on both sides of sickle-shaped recess 6. Clamping or holding points 11 and 21 have short attachment pieces 16, 26 sewn thereto. First strap 10 or second strap 20 are guided adjustably in length through attachment pieces 16, 26. In the same fashion at holding point 31 on the side opposite sickle-shaped recess 6, an attachment piece 36 is sewn on, having a guide ring 32 to which strap 30 is fixed adjustably in length. Three holding points 11, 21 and 31 are arranged at positions on shoulder pad 2 spaced from one another and the straps arranged accordingly thereon each intersect at an angle of approximately 120° in an imaginary elongation.

Straps 10 and 20 are connected to forearm holder 3 or secured there such that when straps 10 and 20 lie twist-free, they cross in the area of points 14 and 24. This crossing point lies below the upper arm and permits additional stabilization of shoulder pad 2. Forearm holder 3, preferably constructed as a closed clasp or here as a textile-like cuff, has an anti-slip feature 7 on its inside which is formed of a silicone pad. The spacing between shoulder pad 2 and forearm holder 3 can be adjusted with the aid of straps 10 and 20. Straps 10 and 20 are fed through guide rings 12 and 22 and after the desired length has been set, can be fixed to straps 10 and 20 using velcro areas 13 and 23 located at the ends. Third strap 30 has a loop 34 in its end region which is positioned such that it runs from shoulder pad 2 behind the back to the opposite shoulder. To adjust the length, third strap 30 is fed through guide ring 32 which is fixed to holding point 31 via attachment piece 36 and is secured to this with the aid of velcro area 33 provided at the end of the third strap.

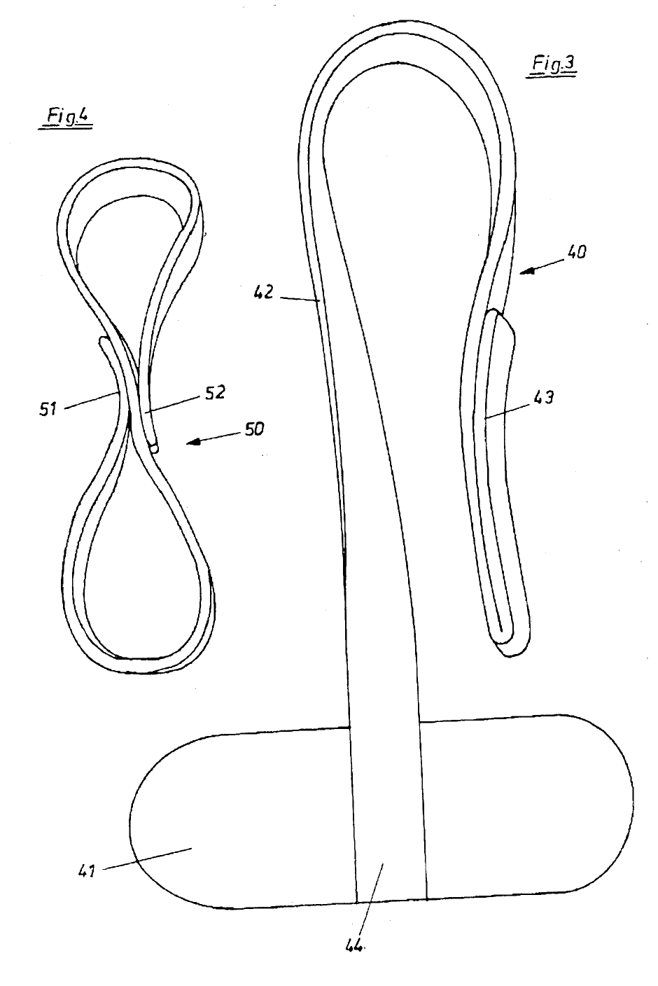

FIG. 3 shows a view of a first holding strap 40. This is fixed to the wrist using a wrist holder 41 which is constructed as a ring-shaped closing wrist cuff and can be closed with a velcro closure. In a central region of wrist holder 41 is a strap 42 which is guided around the back in the elongation of the forearm bent in front of the body and is guided around the upper arm to the injured shoulder and is fixed there with the aid of velcro area 43 in end region of the strap 42. On wrist holder 41, i.e., on the outside of wrist holder 41, is an open flap 44 in continuation or linear extension of strap 42.

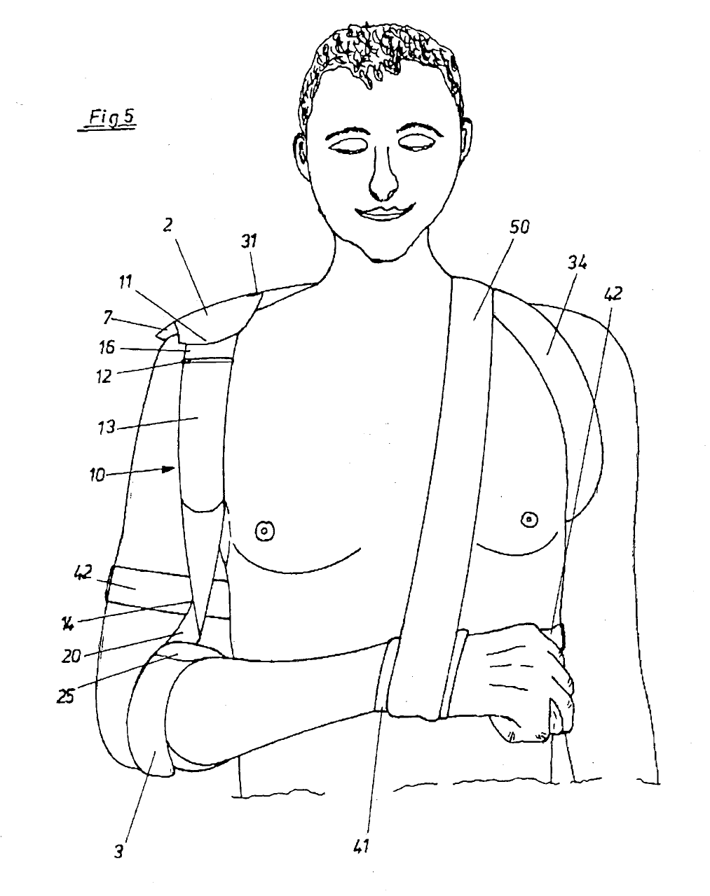

FIG. 4 shows a second holding strap 50 which is also provided to fix the forearm bent in front of the body. An end region 51 of a second holding strap 50 is guided around the vertically aligned forearm or the wrist and can be guided through the then vertically open, horizontally aligned flap 44 so that lateral slippage is prevented. A loop can be formed in this region with the aid of a velcro area provided in end region 51. Another end region 52 of second holding strap 50 also has a velcro area which can be fixed in the region of the shoulder opposite to the shoulder to be treated on third strap 30 of orthesis 1 running therethrough.

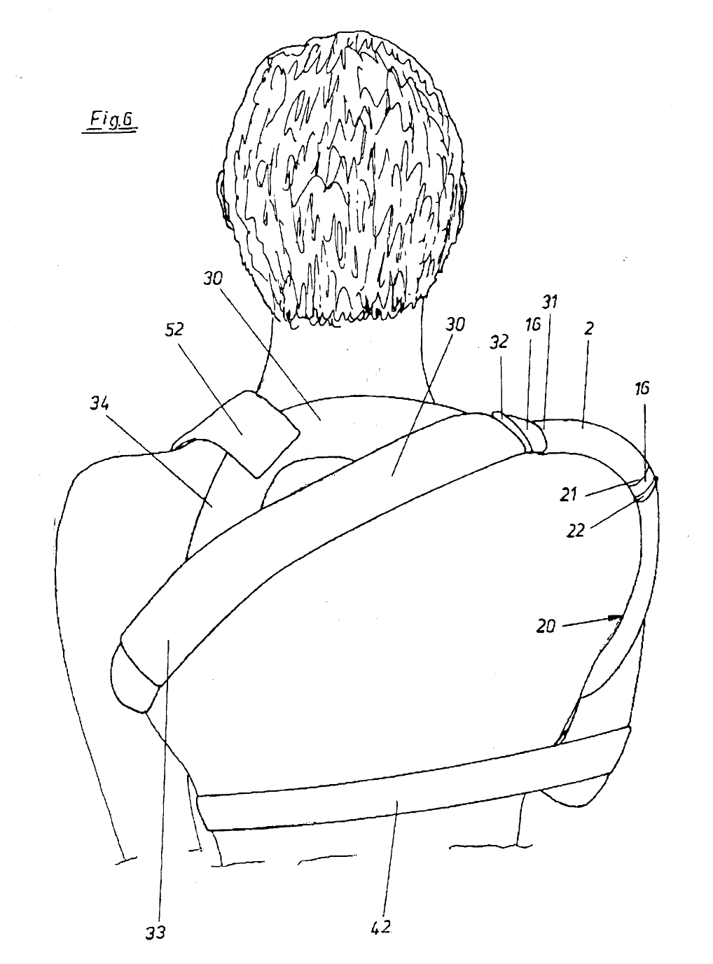

FIG. 5 shows a front view of a preferred embodiment of orthesis 1 according to the invention with first holding strap 40 and second holding straps 50 assigned to the orthesis 1 on the patient. Shoulder pad 2, forearm holder 3 and strap 10 placed at holding point 11 are substantially shown. Starting from holding point 11 there is arranged first attachment piece 16 and then guide ring 12 on shoulder pad 2. Strap 10 is fed through guide ring 12 and fixed with the aid of its velcro area 14 in the end region, adjusted in length. Below the forearm in the vicinity of crossing point 14 first strap 10 crosses second strap 20 which is secured at connection point 25 on forearm holder 3. Sickle-shaped cutaway region 7 can also be seen on shoulder pad 2 above the outside of the shoulder. On the opposite side there is provided holding point 13 for third strap to which ends in loop 34 laid around the opposite shoulder. In order to fix the forearm in front of the body this is fixed using the holding straps 40 and 50. Holding strap 40 has a wrist cuff 41 starting from which strap 41 is placed around the back and guided on the left of the drawing around the opposite upper arm and fixed there with the aid of a velcro closure. Second holding strap 50 connects the wrist with the shoulder opposite the injured shoulder and holds the arm high in this fashion.

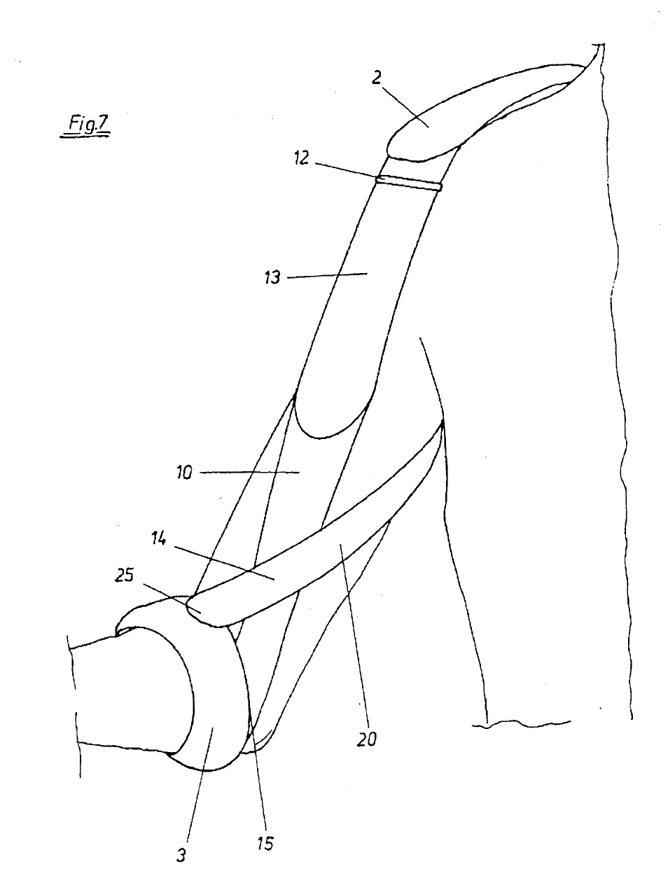

FIG. 6 is a rear view of the orthesis according to the invention positioned as in FIG. 5. Starting from shoulder pad 2, at holding points 21 and 31 are attachment pieces 26 and 36 arranged thereon with guide rings 22 and 32. Second strap 20, starting from guide ring 22, passes behind the back to the forearm holder not visible from this perspective. Third strap 34 placed around the opposite shoulder, is fed through guide ring 32 and adjusted in length. The end region of third strap 30 can be fixed onto strap 30 with velcro area 33 arranged there according to length or it can be fixed to loop 34 as shown here. End 52 of second holding strap 50 is also fixed on third strap 30 or loop 34 with the aid of its velcro connection in this shoulder region. Also seen here is strap 42 of first holding strap 40 which lies around the back and is fixed to the upper arm on the right of the drawing.

FIG. 7 shows an enlarged view in the region of the upper arm which particularly shows the crossing straps. First strap 10 and second strap 20 are secured, especially sewn to forearm holder 3 at connection points 15 and 25 such that when first strap 10 and the second strap 20 are suitably fixed to shoulder pad 2 and first strap 10 and second strap 20 are aligned twist-free, these cross at crossing point 14. As a result of the slip-free fixing of forearm holder 3, this application or fixing of straps 10 and 20 at points 15 and 25 on the forearm holder 3 at some distance apart results in a particularly secure fit of the orthesis.

Accordingly, while at least one embodiment of the present invention has been shown and described, it is to be understood that many changes and modifications may be made thereunto without departing from the spirit and scope of the invention as defined in the appended claims.

What is claimed is:

1. An orthesis for the treatment of injuries of the acrominioclavicula joint (A-C joint) in patients comprising:
a shoulder pad disposed on a shoulder of the patient closest to the injured A-C joint;
a forearm holder disposed around a forearm in front of an elbow joint of the patient below said shoulder closest to the injured A-C joint;
a first strap, having a first end secured to said forearm holder and a second end secured to said shoulder pad in front of said shoulder;
a second strap, having a first end secured to said forearm holder and a second end secured to said shoulder pad behind said shoulder; and
a third strap, having a first end secured to said shoulder pad and a second end having a loop, wherein said third strap runs behind a back of the patient to an opposite shoulder where said second end of said third strap loops around said opposite shoulder.

2. The orthesis according to claim 1, further comprising an anti-slip silicone support on a side of said shoulder pad facing the patient or the skin of the patient.

3. The orthesis according claim 1, wherein said shoulder pad is made of a textile material.

4. The orthesis according claim 1, wherein said first strap, said second strap and said third strap are detachably connected to said shoulder pad.

5. The orthesis according to claim 1, further comprising at least three holding points of said shoulder pad spaced apart from one another, wherein said at least three holding points secure said first strap, said second strap and said third strap.

6. The orthesis according to claim 1, wherein said forearm holder is constructed as a forearm clasp.

7. The orthesis according to claim 1, wherein said first strap and said second strap are secured to said shoulder pad and said forearm holder so that when said first strap and said second strap are applied twist-free, they are aligned so that they cross under an upper arm of the patient.

8. The orthesis according to claim 1, further comprising an anti-slip feature disposed on an inside of said forearm holder.

9. The orthesis according to claim 1, wherein said loop on said third strap may be opened with a velcro fastening.

10. The orthesis according to claim 1, further comprising a first holding strap secured on a wrist of the patient and guided in a direction out from said forearm bent in front of the body of the patient, around said back of the patient, and secured on said upper arm of said injured shoulder.

11. The orthesis according to claim 10, further comprising a second holding strap secured on a wrist of the patient and guided upwards with said forearm bent in front of the body and secured there on said third strap in the region of said opposite shoulder.

12. The orthesis according to claim 11, wherein said first holding strap has a wrist holder and said second holding strap is secured on said wrist holder.

13. The orthesis according to claim 11, wherein said second holding strap has a wrist holder and said first holding strap is secured on said wrist holder.
